(19) United States
(12) Patent Application Publication
(10) Pub. No.: US 2007/0233123 A1
(43) Pub. Date: Oct. 4, 2007

(54) Bone fixation device

(75) Inventors: Shaher A. Ahmad, Plano, TX (US); Kerry Zang, Paradise Valley, AZ (US)

Correspondence Address: BAKER BOTTS L.L.P., 2001 ROSS AVENUE, SUITE 600, DALLAS, TX, 75201-2980, US

(73) Assignee: OsteoMed, L.P.

(21) Appl. No.: 11/359,244

(22) Filed: Feb. 21, 2006

(51) Int. Cl. A61B 17/58

(52) U.S. Cl. 606/73

(57) ABSTRACT

A bone fixation device includes an elongate shaft including a first end and a second end, the first end generally opposite the second end. The elongate shaft defines a hollow longitudinal bore and at least one opening passing from the hollow longitudinal bore through a sidewall of the elongate shaft. The fixation device also includes a section of threads proximate the first end.

### TECHNICAL FIELD OF THE INVENTION

This invention relates generally to medical devices and more specifically to a bone fixation device.

### BACKGROUND

Following a bone fracture or operative procedure, portions of a bone may need to be fixed to one another to provide proper alignment of the bone portions and to promote healing. A bone screw may be used to couple the portions of the bone together. The bone screws may hold the portions of bone in a particular orientation relative to one another and allow for growth of new bone between the two bones. The newly grown bone may fuse the portions of bone together. During the time that the new bone is growing between the bone portions, the bone screw may hold and support the bone portions in the proper position.

Particular bone screws may include a first threaded portion in which a thread pitch is greater than a thread pitch of a second threaded portion. Between the first and second threaded portions may be an unthreaded section. The first section of threads having the greater thread pitch may be screwed through a first portion of bone and into a second portion of bone. As the second portion of threads with the lesser thread pitch is being threaded into the first portion of bone, the second portion of bone may be pulled towards the first portion of bone. The movement of the second portion of bone towards the first portion of bone is caused by the greater thread pitch of the first portion of threads relative to the second portion of threads. The greater thread pitch draws the second portion of bone towards the first portion of bone because the first portion of threads will be embedding into the second portion of bone at a greater rate than the second portion of threads will be embedding into the first bone portion. This results in a relative movement of the bone portions as the first portion of bone moves relative to the bone screw at a greater rate than the first portion of bone. Bone screws of this type may be used to reduce open spaces between bones allowing the bones to heal more quickly. The first and second portions of bone may also be held more securely as portions of the first and second sections of bones may abut.

When implanting a bone screw into a portion of bone, a countersink hole may be drilled such that the head of the bone screw does not protrude above a surface of the bone. Countersinking the bone screw prevents soft tissue irritation from the head of the screw which might otherwise protrude above the bone surface. Bone screws may need to be installed at a variety of angles, and the countersink hole for the head of the bone screw would need be drilled accordingly.

### SUMMARY

In accordance with particular embodiments of the present invention, a bone screw includes an elongate shaft including a first end and a second end, the first end generally opposite the second end. The elongate shaft defines a hollow longitudinal bore and at least one opening passing from the hollow longitudinal bore through a sidewall of the elongate shaft. The bone screw also includes a section of threads proximate the first end.

In a particular embodiment, a biological material may be injected into the hollow longitudinal bore such that a portion of the biological material passes through the opening. In some embodiments, the biological material may include bone substitute, bone cement, infection preventative, biologics, antibiotics, and bone morphogenic proteins.

In another embodiment, a bone screw may include an elongate shaft including a first end and a second end, the first end generally opposite the second end. The bone screw may also include a section of threads coupled to the elongate shaft and extending from the second end along at least a portion of a length of the elongate shaft. Threads of the section of threads proximate the second end are beveled such that the bone screw may be implanted flush with a surface of a bone into which the bone screw is being implanted when the bone screw is being implanted at an angle to the surface of the bone.

Certain embodiments of the present invention may also include a method of joining bones including implanting a bone screw into first and second bones to join the bones. The bone screw may include an elongate shaft with a first end and a second end. The first end generally opposite the second end. The elongate shaft may also define a hollow longitudinal bore. The bone screw may also include a section of threads. The method may also include injecting a biological material into the longitudinal bore until the biological material exudes from a opening passing from the hollow longitudinal bore through a sidewall of the elongate shaft.

Technical advantages of particular embodiments of the present invention may include the ability to inject biological material through an end of a bone fixation device, such as a bone screw, into a volume surrounding the bone fixation device. When the bone fixation device is coupling two portions of bone, the biological material injected through the bone fixation device may fill the cracks and voids remaining in and between the two bone portions. The injected biological material may include a material designed to aid in the fixation of the bone portions, aid in the healing of the bone portions, or to prevent infection of the site adjacent the bone fixation device.

Another technical advantage of certain embodiments of the present invention may include a screw that is completely implanted in the bone portions and does not extend above the surface of the bone portions. An end of the bone screw may be un-flared and beveled such that the screw may be completely implanted either perpendicular to the bone portions or at a variety of angles. A threaded screw head without flares may allow the screw to be completely implanted perpendicular to the bones without the need for drilling a countersink hole. Beveling the threads may allow the bone screw to be completely implanted in the bone at an angle without a countersink hole. The ability to completely implant the bone screw without drilling a countersink hole reduces patient trauma and increases the strength of the surrounding bone.

Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

To provide a more complete understanding of the present invention and the features and advantages thereof, reference is made to the following description, taken in conjunction with the accompanying drawings, in which:

FIG. 1 illustrates a cannulated bone screw in accordance with one embodiment of the present invention;

FIG. 2 illustrates a side view of the bone screw of FIG. 1 in accordance with a particular embodiment of the present invention;

FIG. 3 illustrates an end view of the bone screw of FIG. 1 in accordance with an embodiment of the present invention;

FIG. 4 illustrates the bone screw of FIG. 1 as it may be implanted into a fractured bone;

FIG. 5 illustrates a perspective view of a screw in accordance with another embodiment of the present invention;

FIG. 6 illustrates a longitudinal cross section of the screw of FIG. 5 in accordance with a particular embodiment of the present invention;

FIG. 7 illustrates a perspective view of a dowel in accordance with another embodiment of the present invention; and

FIG. 8 illustrates a longitudinal cross section of the dowel of FIG. 7 in accordance with a particular embodiment of the present invention.

### DESCRIPTION OF EXAMPLE EMBODIMENTS

Particular embodiments of the present invention provide a bone fixation device for coupling together first and second segments of bone. The bone fixation device includes a hollow longitudinal bore and openings passing from the hollow longitudinal bore to the exterior of the bone fixation device through the sidewalls of the bone fixation device. The openings allow injection of biological materials through an end of the bone fixation device through the openings to the site surrounding the bone fixation device. In certain embodiments, the bone fixation device may be a threaded bone screw. The bone screw may include a beveled portion proximate one end that allows the bone screw to be completely implanted into bone when the bone screw is implanted at an angle.

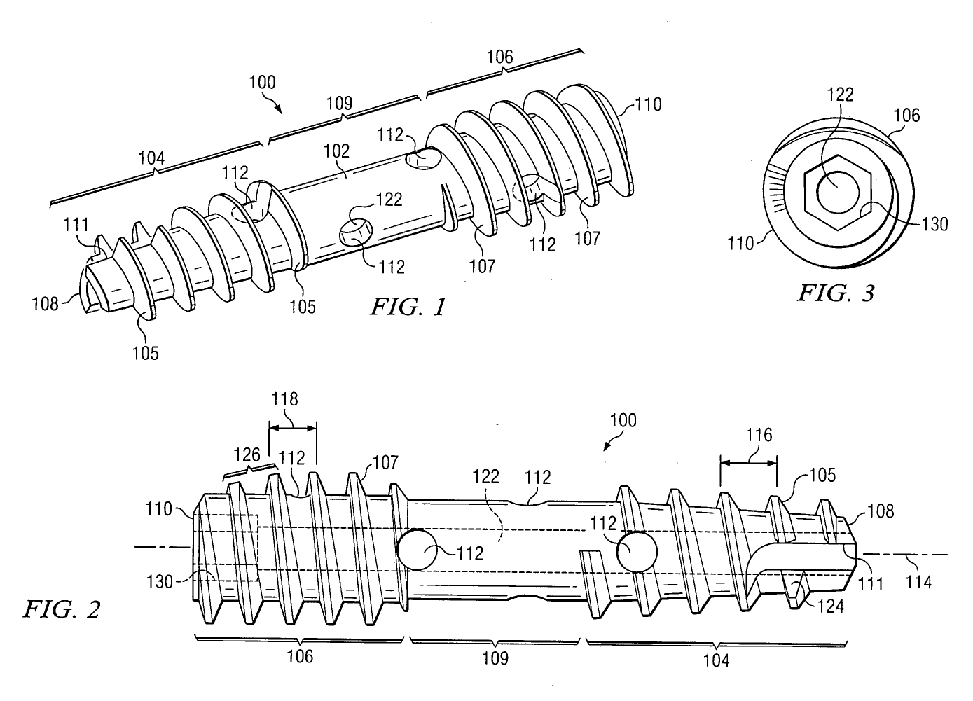

FIGS. 1-3 illustrate a bone fixation device in accordance with a particular embodiment of the present invention. The bone fixation device illustrated in FIGS. 1-3 is a bone screw 100. Bone screw 100 includes a first section of threads 104 and a second section of threads 106. First section of threads 104 includes threads 105, and second section of threads 106 includes threads 107. Between first section of threads 104 and second section of threads 106 is an unthreaded section 109. When implanted in a patient, first section of threads 104 may couple a first segment of bone to bone screw 100 and second section of threads 106 may couple a second portion of bone to bone screw 100.

Bone screw 100 may also include a first end 108 and a second end 110. First end 108 may comprise a drilling point to aid in implanting bone screw 100 into first and second segments of bone. In the embodiment illustrated, first end 108 includes a self-tapping head 124 and a cutting flute 111. Second end 110 may include a rotational interface 130, such as a hexagonal or other interface, to allow a tool, such as a drill, to interface with bone screw 100 for the purpose of rotating bone screw 100 and implanting bone screw 100 into first and second bone segments.

Bone screw 100 may also include shaft 102. In the embodiment illustrated, shaft 102 generally includes a cylindrical portion from end 110 to the portion of the shaft at which threads 104 begin and a conical portion that generally narrows from the portion of the shaft at which threads 104 begin to end 108. However, in other embodiments, shaft 102 may include one or more portions having any suitable shape or combination of shapes. Shaft 102 as well as threads 105 and 107 may have a plurality of openings 112 removed from them. Openings 112 may pass through the outer wall of shaft 102 and intersect a hollow longitudinal bore 122 of bone screw 100. The hollow longitudinal bore 122 may intersect with the rotational interface 130 accessible from second end 110. This design allows a biological material to be injected through second end 110 through rotational interface 130 through longitudinal bore 122 and out of openings 112 into the environment surrounding bone screw 100. When bone screw 100 is coupling two portions of bone, the biological material injected through bone screw 100 may fill the cracks and voids remaining in and between the two bone portions. The injected biological material may include a material designed to aid in the fixation of the bone portions, aid in the healing of the bone portions, or to prevent infection of the site adjacent bone screw 100.

The injection of biological material through openings 112 may generally occur after bone screw 100 has been implanted into first and second bone segments. In particular embodiments, bone screw 100 may come pre-filled with a biological material, or bone screw 100 may be primed prior to implantation into first and second bone segments. Priming may include filling some or all of longitudinal bore 122 and openings 112 with a biological material to be injected following implantation of bone screw 100. The biological material may include any mixture of fluids, dissolved solids, or particulate solids that may assist in healing the bone segments. In particular embodiments, the biological material may include one or more of a bone substitute, demineralized bone matrix, cancellous bone chips, a bone cement, an infection preventative, biologics, antibiotics, and bone morphogenic proteins. In certain embodiments, a biological material including more of the above materials may be injected to fill the volume surrounding bone screw 100.

In the embodiment illustrated, a thread pitch 116 of threads 105 is greater than a thread pitch 118 of threads 107. A thread pitch is a distance between crests of adjacent threads. For example, thread pitch 116 is the distance between adjacent threads 105. Having a greater thread pitch in first portion of threads 104 than second portion of threads 106 enables bone screw 100 to draw together first and second portions of bone. Bone screw 100 is able to draw together first and second portions of bone because first section of threads 104 will engage a second portion of bone and draw it towards second section of threads 106, which is engaging a first portion of bone. Some embodiments may not include threads having different thread pitches, and some embodiments may include only a singe section of threads or no threads at all.

In practice, bone screw 100 may be rotated to implant bone screw 100 into first and second portions of bone. First portion of threads 104 will first pass through a first portion of bone. First portion of threads 104 will then engage a second portion of bone as second portion of threads 106 engages the first portion of bone. The greater thread pitch of first portion of bone 104 will cause a movement of the first portion of bone parallel to longitudinal axis 114 of bone screw 100 in the direction of second end 110. The movement of the first portion of bone relative to the second portion of bone may continue until the second section of threads 106 is implanted into the first portion of bone and rotation of bone screw 100 ceases.

Drawing together the portions of bone coupled by bone screw 100 minimizes the voids or open spaces between the first and second portions of bone. The smaller the gap between the first and second portions of bone, the faster new bone will be able to grow between and couple the first and second portions, thereby healing the separated bone segments. To further increase the rate of healing, the void between the first and second bone segments may be filled with the injection biological material. The injection biological material may fill at least a portion of the remaining voids and speed the rate of new bone growth as well as the strength of the resulting bone.

In the illustrated embodiment, second section of threads 106 includes a beveled portion 126. Beveled portion 126 may be adjacent second end 110 and may include portions of threads 107 closest to second end 110. Beveled portion 126 may allow bone screw 100 to be implanted at an angle to enable the threads to engage the more dense portion of the bone while preventing threads 107 adjacent second end 110 from protruding above the surface of the bone segment in which second portion of threads 106 is implanted. The angle and depth of beveled portion 126 may vary depending on a desired angle of implantation of bone screw 100. In particular embodiments, the angle of bevel of beveled portion 126 may be up to 45 degrees. Moreover, a depth of rotational interface 130 may be selected such that the ability to rotate bone screw 100 using rotational interface 130 will not be hampered by the angle or depth of beveled portion 126. In particular embodiments, the beveled portion corresponds to only the threads such that the head of the screw (e.g., at end 110) is not beveled. Thus, the bone screw may have a full, unbeveled head or end while also having a beveled portion of threads at the end which enable the threads to engage the more dense portion of the bone and which do not protrude above the surface of the bone segment when the bone screw is implanted at an angle.

FIGS. 2 and 3 illustrate rotational interface 130 as a hexagonally shaped recess. However, rotational interface 130 may be potentially any shape allowing the interface of bone screw 100 with a rotational tool. In particular embodiments, the shape of rotational interface 130 may be square, rectangular, triangular, star shaped, or cross shaped.

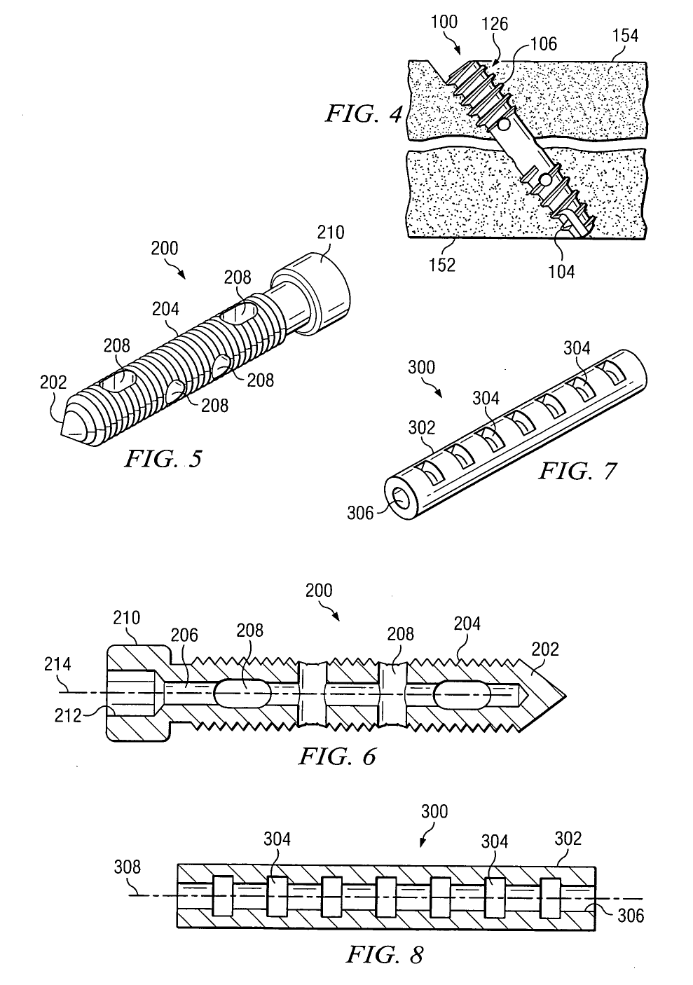

FIG. 4 illustrates bone screw 100 as it may be implanted into first and second portions of bone 152 and 154. First section of threads 104 may be implanted into bone 152, and second section of threads 106 may be implanted into bone 154. As illustrated, beveled portion 126 allows the second section of threads 106 to be flush with the exterior surface of bone 154 without countersinking bone screw 100. Bone screw 100 has been implanted into bones 152 and 154 at an angle relative to the surface of bone 154. If second portion of threads 106 did not include beveled portion 126, second section of threads 106 would need to be implanted further into bone 154 to avoid complications caused by exposed threads. This would result in less engagement between second portion of threads 106 and bone 154. Therefore, beveled portion 126 allows for maximum engagement of second section of threads 106 with bone 154 without the potential for complications caused by leaving threads exposed above the surface of bone 154.

A bone fixation device incorporating aspects of the present invention may include any dimensions appropriate for an intended application. In particular embodiments, a bone fixation device may have a diameter of approximately 3 to 9 millimeters and a length between 10 and 140 millimeters. In a specific embodiment, a bone screw may have a diameter of 3 millimeters and a length of 120 millimeters.

FIGS. 5 and 6 illustrate a screw 200 in accordance with a particular embodiment of the present invention. In some embodiments, screw 200 may be a bone screw. Screw 200 includes an elongate shaft 202 and a plurality of threads 204 arranged around the elongate shaft 202. While threads 204 have been illustrated along the entire length of elongate shaft 202, any portion of elongate shaft 202 may be threaded or unthreaded, or elongate shaft 202 may include multiple sections of threads like bone screw 100. In particular embodiments, elongate shaft 202 may not include any threads 204.

Screw 200 also includes a head 210 that defines a rotational interface 212. Rotational interface 212 may be similar to rotational interface 130 discussed above with reference to FIGS. 1-3. Elongate shaft 202 defines a hollow longitudinal bore 206 parallel to a longitudinal axis 214 of screw 200. Hollow longitudinal bore 206 extends from rotational interface 212 through a length of elongate shaft 202. Longitudinal bore 206 is illustrated as terminating before exiting the end of elongate shaft 202 opposite rotational interface 212. In particular embodiments, longitudinal bore 206 may pass through the end of elongate shaft 202 opposite rotational interface 212.

Intersecting longitudinal bore 206 are a plurality of openings 208. Openings 208 may intersect longitudinal bore 206 and extend from longitudinal bore 206 through a sidewall of elongate shaft 202. Openings 208 may allow biological materials to pass from longitudinal bore 206 into the volume surrounding screw 200. The biological materials may include any suitable biological material, such as those discussed above with reference to FIGS. 1-3. The biological materials may be injected through rotational interface 212, down longitudinal bore 206, and out openings 208 to fill the voids or openings surrounding screw 200. While openings 208 have been illustrated as having a oval shape, a shape of openings 208 is not essential, and openings 208 may have any suitable shape.

FIGS. 7 and 8 illustrate a dowel 300 in accordance with a particular embodiment of the present invention. Dowel 300 includes an elongate shaft 302 that defines a hollow longitudinal bore 306 parallel to longitudinal axis 308 of dowel 300. Elongate shaft 302 also defines a plurality of openings 304 that intersect hollow longitudinal bore 302 and pass through a sidewall of elongate shaft 302. Dowel 300 may be implanted into a section of bone or may be used to couple together two or more sections of bone. Dowel 300 may be implanted into an existing hole, or a hole may be drilled for dowel 300. Dowel 300 may then be pressed or hammered into the hole. A biological material may then be injected through longitudinal bore 306 and pass through openings 304 into the volume surrounding dowel 300. The injected biological material may include any suitable biological material, such as those discussed above regarding FIGS. 1-3. Openings 304 have been illustrated as having a roughly rectangular shape, however, openings 304 may be any suitable shape. Furthermore, dowel 300 has been illustrated as having a cylindrical shape, but dowel 300 may be conically shaped or may have any other desired shape. The ends of dowel 300 may also be rounded or beveled to ease the insertion of dowel 300 into bone or to provide a flush implantation when dowel 300 is implanted at an angle.

Although the present invention has been described with several embodiments, a number of changes, substitutions, variations, alterations, and modifications may be suggested to one skilled in the art, and it is intended that the invention encompass all such changes, substitutions, variations, alterations, and modifications as fall within the spirit and scope of the appended claims.

What is claimed is:

1. A fixation device, comprising:
an elongate shaft including a first end and a second end, the first end generally opposite the second end, the elongate shaft defining a hollow longitudinal bore, the elongate shaft also defining at least one opening passing from the hollow longitudinal bore through a sidewall of the elongate shaft; and
a section of threads proximate the first end.

2. The fixation device of claim 1, wherein the longitudinal bore is configured to receive a biological material injected into the longitudinal bore such that a portion of the biological material passes through the opening.

3. The fixation device of claim 2, wherein the biological material comprises one or more materials selected from the group consisting of: bone substitute, bone cement, infection preventative, biologics, antibiotics, and bone morphogenic proteins.

4. The fixation device of claim 1:
wherein the section of threads comprises a first section of threads; and
further comprising a second section of threads proximate the second end, the second section of threads including a beveled portion proximate the second end that enables implantation of the fixation device approximately flush with a surface of a bone into which the fixation device is implanted when the fixation device is implanted at an angle to the surface of the bone.

5. The fixation device of claim 1:
wherein the section of threads comprises a first section of threads; and
further comprising a second section of threads proximate the second end, wherein the second section of threads comprises threads that are spaced more closely together than threads of the first portion of threads.

6. The fixation device of claim 5, further comprising an unthreaded portion between the first section of threads and the second section of threads such that the first portion of threads may engage a first bone and draw the first bone toward a second bone being engaged by the second portion of threads.

7. The fixation device of claim 1, wherein the elongate shaft comprises a conical shape.

8. The fixation device of claim 7:
wherein the section of threads comprises a first section of threads; and
further comprising a second section of threads proximate the second end, the second section of threads extending radially outwards from a longitudinal axis of the elongate shaft further than the first section of threads.

9. A fixation device, comprising:
an elongate shaft including a first end and a second end, the first end generally opposite the second end;
a section of threads coupled to the elongate shaft and extending from the second end along at least a portion of a length of the elongate shaft;
wherein threads of the section of threads proximate the second end are beveled such that the fixation device may be implanted approximately flush with a surface of a bone into which the fixation device is implanted when the fixation device is being implanted at an angle to the surface of the bone; and
wherein the second end is unbeveled.

10. The fixation device of claim 9, wherein the elongate shaft defines a hollow longitudinal bore and at least one opening passing from the hollow longitudinal bore through a sidewall of the elongate shaft.

11. The fixation device of claim 10, wherein the longitudinal bone is configured to receive a biological material injected into the longitudinal bore such that a portion of the biological material passes through the opening.

12. The fixation device of claim 11, wherein the biological material comprises one or more materials selected from the group consisting of: bone substitute, bone cement, infection preventative, biologics, antibiotics, and bone morphogenic proteins.

13. The fixation device of claim 9:
wherein the section of threads comprises a first section of threads; and
further comprising a second section of threads proximate the first end, wherein the first section of threads comprises threads that are spaced more closely together than threads of the second portion of threads.

14. The fixation device of claim 13, further comprising an unthreaded portion between the first section of threads and the second section of threads such that the first portion of threads may engage a first bone and draw the first bone toward a second bone being engaged by the second portion of threads.

15. The fixation device of claim 9, wherein the elongate shaft comprises a conical shape.

16. The fixation device of claim 15:
wherein the section of threads comprises a first section of threads; and
further comprising a second section of threads proximate the first end, wherein the first portion of threads extend radially outwards from a longitudinal axis of the elongate shaft further than the second portion of threads.

17. A fixation device, comprising:
an elongate shaft including a first end and a second end, the first end generally opposite the second end;
the elongate shaft defining a hollow longitudinal bore;
the elongate shaft also defining at least one opening passing from the hollow longitudinal bore through a sidewall of the elongate shaft; and
wherein a biological material may be injected into the hollow longitudinal bore such that a portion of the biological material passes through the opening.

18. The fixation device of claim 17, wherein the biological material comprises one or more materials selected from the group consisting of: bone substitute, bone cement, infection preventative, biologics, antibiotics, and bone morphogenic proteins.

19. A method of joining bones, comprising:
implanting a fixation device into first and second bones to join the bones, the fixation device including:
an elongate shaft with a first end and a second end, the first end generally opposite the second end, wherein the elongate shaft defines a hollow longitudinal bore;
a section of threads proximate the first end; and
injecting a biological material into the longitudinal bore until the biological material exudes from a opening passing from the hollow longitudinal bore through a sidewall of the elongate shaft.

20. The method of claim 19, wherein the biological material comprises one or more materials selected from the group consisting of: bone substitute, bone cement, infection preventative, biologics, antibiotics, and bone morphogenic proteins.

21. The method of claim 19, wherein:
implanting the fixation device into the first and second bones comprises implanting the fixation device at an angle relative to a surface of the first bone at a point there the fixation device enters the first bone; and
the section of threads comprise beveled threads adjacent the first end such that the fixation device may be implanted approximately flush with the surface of the first bone.
